(19) United States
(12) Patent Application Publication
(10) Pub. No.: US 2021/0000442 A1
(43) Pub. Date: Jan. 7, 2021

(54) METHOD AND SYSTEM FOR INDICATING THE LIKELIHOOD OF A GASTROINTESTINAL CONDITION

(71) Applicant: The University of Western Australia, Crawley (AU)

(72) Inventors: Barry James Marshall, Shenton Park (AU); Katherine Mary Webberley, Cottesloe (AU); Gary Andrew Peter Allwood, Heathridge (AU); Du Xuhao, Shenton Park (AU); Wenchao Wan, Shenton Park (AU); Adam Osseiran, Shelley (AU)

(73) Assignee: The University of Western Australia, Crawley (AU)

(21) Appl. No.: 16/969,543

(22) PCT Filed: Dec. 13, 2018
(86) PCT No.: PCT/AU2018/051332

§ 371 Date: Aug. 12, 2020

(30) Foreign Application Priority Data
Feb. 14, 2018 (AU) 2018900459

(51) Int. Cl. A61B 7/00; G06F 17/18

(52) U.S. Cl. CPC A61B 7/008; G06F 17/18

(57) ABSTRACT

A system (10) for indicating the likelihood of a gastrointestinal (GI) condition by analysing bowel sounds is provided, the system (10) comprising a sound detector (12) configured to detect bowel sounds and generate a corresponding signal representative of the bowel sounds, a signal processor arranged to identify a plurality of bowel sound signals within the corresponding signal, each bowel sound signal representative of an individual bowel sound, wherein the system is arranged to identify at least one feature from each of the plurality of bowel sound signals so as to produce a collection of values for the same at least one feature, and determine at least one statistical distribution property of the collection of values, the statistical distribution property capable of at least assisting in providing an indication of the existence or non-existence of a GI condition, and wherein the system is further arranged to associate the at least one statistical distribution property with a reference parameter and determine the likelihood of the GI condition based on the association. A corresponding method is also provided.

### TECHNICAL FIELD

The present invention relates to a method and a system for indicating the likelihood of a gastrointestinal condition, and more particularly, although not exclusively, to a method and system for indicating the likelihood of a functional gastrointestinal disorder such as irritable bowel syndrome, and/or the likelihood of a gastrointestinal organic disease, such as inflammatory bowel disease and/or differentiation between the two.

### BACKGROUND

Functional gastrointestinal (GI) disorders such as irritable bowel syndrome (IBS), and GI organic diseases such as inflammatory bowel disease (IBD) including Crohn's disease and ulcerative colitis are debilitating GI conditions. They can also be common, IBS for example is estimated to affect around 11% of the world's population.

The current gold standard for IBS diagnosis is through the Rome IV symptom based diagnostic criteria. While offering positive diagnosis, these criteria do not have high reliability (low sensitivity). Physicians typically diagnose IBS through a process of exclusion, i.e., ruling out a number of organic diseases that share symptoms with IBS. Initial screening would usually include baseline blood tests and stool tests for exclusion of infections, coeliac disease and IBD. Typically, primary care physicians also refer patients for colonoscopy and biopsy, even though colonoscopy has been found to reveal a GI organic disease, such as IBD in only a small percentage of patients with IBS symptoms.

These invasive tests are a burden to health systems, contributing to lengthening waiting lists for gastroenterological review as well as adding to the financial costs associated with IBS. Colonoscopies are not only unpleasant for patients but carry significant risks. In addition to these risks, the burden on patients is multifaceted including physical discomfort, psychological distress, and financial costs due to time off-work. Further, since IBS is unrelated to any obvious structural or biochemical changes in the gut, these invasive procedures cannot provide a positive diagnosis for IBS. A diagnosis of exclusion often leaves patients confused and reluctant to engage in treatment. A cost-effective test that could provide a positive diagnosis for patients with a family or symptom history of IBS would be hugely beneficial in diagnosis and overall management of the condition.

In addition, for patients who have a GI organic disease, such as IBD or coeliac disease, a non-invasive test would be an extremely useful and cost-effective screening tool, prior to confirmation with biopsy.

There is a need for a new cost-effective, accurate and non-invasive diagnostic test for gastrointestinal conditions.

### SUMMARY

It would be advantageous if a non-invasive test could allow determining a likelihood of an individual having a GI condition versus having healthy bowels. It would further be advantageous if a single non-invasive test could allow (i) differentiating between healthy individuals and individuals suffering from a functional GI disorder such as IBS, (ii) differentiating between healthy individuals and individuals suffering from a GI organic disease such as IBD, and (iii) differentiating between individuals suffering from a functional GI disorder and individuals suffering from a GI organic disease. Thus, it would be advantageous if a single non-invasive test could allow indicating a likelihood of an individual having a functional GI disorder versus being healthy, a likelihood of an individual having a GI organic disease versus being healthy, and a likelihood of the individual having a functional GI disorder versus having a GI organic disease.

In broad terms, embodiments of the present invention seek to provide an indication of a likelihood that a patient may have a GI condition or may have healthy bowels based on the patient's bowel sounds. This may provide a cost-effective and non-invasive diagnostic test for a GI condition, including a functional GI disorder such as IBS and a GI organic disease such as IBD.

According to a first aspect of the invention, there is provided a system for indicating a likelihood of a gastrointestinal (GI) condition by analysing bowel sounds, the system comprising:
a sound detector configured to detect bowel sounds and generate a corresponding signal representative of the bowel sounds;
a signal processor arranged to identify a plurality of bowel sound signals within the corresponding signal, each bowel sound signal representative of an individual bowel sound;
wherein the system is arranged to identify at least one feature from each of the plurality of bowel sound signals so as to produce a collection of values for the same at least one feature, and determine at least one statistical distribution property of the collection of values, the at least one statistical distribution property being capable of at least assisting in providing an indication of the existence or non-existence of a GI condition; and
wherein the system is further arranged to associate the at least one statistical distribution property with a reference parameter and determine the likelihood of the GI condition based on the association.

The at least one statistical distribution property may comprise skewness and/or kurtosis.

The system may be arranged to generate an index value based on the association of the at least one statistical distribution property with the corresponding reference parameter, and compare the index value to a threshold value, in order to determine the likelihood of the GI condition.

The system may be arranged to generate the index value using the formula,

$$f = \frac{1}{1 + {e}^{- Σ {c}_{{i}^{x} i}}}$$

where ‘f’ is the index value, ‘xᵢ’ represents each one of the at least one features, ‘i’ is an integer from 1 to n, where n is the number of features, and ‘cᵢ’ represents the reference parameter associated with the feature ‘xᵢ’.

The reference parameter may be a weight value applicable to the associated statistical distribution property of the at least one identified feature.

In one embodiment, the GI condition is a functional GI disorder such as irritable bowel syndrome (IBS). The system may be arranged to determine a likelihood of IBS versus healthy bowels based on the association.

In another embodiment, the GI condition is a GI organic disease such as inflammatory bowel disease (IBD). The system may be arranged to determine a likelihood of IBD versus healthy bowels based on the association.

In a further embodiment, the GI condition includes a functional GI disorder and a GI organic disease, wherein the at least one statistical distribution property is capable of at least assisting in providing an indication of the existence or non-existence of the functional GI disorder and the GI organic disease. The system may be arranged to determine a likelihood of IBS versus IBD based on the association of the at least one statistical distribution property with a corresponding reference parameter.

The system may also be arranged to simultaneously determine at least one statistical distribution property capable of at least assisting in providing an indication of the existence or non-existence of IBS, and at least one statistical distribution property capable of at least assisting in providing an indication of the existence or non-existence of IBD, whereby the system is arranged to simultaneously determine a likelihood of IBS versus healthy bowels and a likelihood of IBD versus healthy bowels based on respective associations of the at least one statistical distribution property with corresponding reference parameters.

The system may further be arranged to determine a likelihood of IBS versus IBD when the respective association of the at least one statistical distribution property with the corresponding reference parameter indicates that IBS is more likely than healthy bowels.

Alternatively, or additionally, the system may also be arranged to determine a likelihood of IBS versus IBD when the respective association of the at least one statistical distribution property with the corresponding reference parameter indicates that IBD is more likely than healthy bowels.

The at least one feature may comprise, or be based on, one or more of the following: amplitude; burst amount; burst ratio; contraction interval time; higher order zero crossing; band energy ratio; spectral bandwidth double frequency; flatness; spectral centroid; energy; dynamic range; mel width; envelope crest factor; and roll off.

In one embodiment, the system is arranged to identify a plurality of different features from each of the plurality of bowel sounds signals and determine the likelihood of the GI condition based on a combination of the different features.

In one embodiment, the system is arranged to determine the likelihood of IBS versus healthy bowels based on a first combination of the different features comprising at least one feature based on: burst; spectral bandwidth double frequency; contraction interval time; or higher order zero crossing.

In another embodiment, the system is arranged to determine the likelihood of IBD versus healthy bowels based on a second combination of the different features comprising at least one feature based on: flatness 3000; or spectral centroid.

The system may also be arranged to determine the likelihood of IBS vs IBD based on a third combination of the different features comprising at least one feature based on: envelope crest factor; or roll off.

The system may be arranged to determine a plurality of different statistical distribution properties of the collection of values for the at least one feature and determine the likelihood of the GI condition based on a combination of the different statistical distribution properties.

The sound detector may comprise at least two acoustic sensors locatable in proximity to an abdominal region of a subject and spaced-apart from each other for detecting bowel sounds from the abdominal region.

The system may be further arranged such that for each bowel sound signal identified by the system, the system identifies one of the at least two acoustic sensors to be associated with the bowel sound signal based on which sensor produced a highest amplitude reading corresponding to the bowel sound signal.

In order to identify individual bowel sound signals, the signal processor may be arranged to divide the corresponding signal into a plurality of segments and, for each segment, determine whether there is a signal portion within any one of the following ranges: 200 Hz to 800 Hz; 600 Hz to 1000 Hz; 800 Hz to 1200 Hz; 1000 Hz to 1600 Hz; and 1600 Hz to 2000 Hz.

According to a second aspect of the invention, there is provided a method of indicating a likelihood of a GI condition by analysing bowel sounds, the method comprising:
obtaining a signal representative of a sound including a plurality of bowel sounds originating from an abdominal region;
identifying a plurality of bowel sound signals within the signal, each bowel sound signal representative of an individual bowel sound;
identifying at least one feature of each of the plurality of bowel sound signals within the signal so as to produce a collection of values for the same at least one feature;
determining at least one statistical distribution property of the collection of values, the statistical distribution property capable of at least assisting in providing an indication of the existence or non-existence of a GI condition;
associating the at least one statistical distribution property with a reference parameter; and
determining the likelihood of the GI condition based on the association.

The at least one statistical distribution property may comprise skewness and/or kurtosis.

The method may comprise generating an index value based on the association of the at least one statistical distribution property with the corresponding reference parameter, and comparing the index value to a threshold value, in order to determine the likelihood of the GI condition.

The method may comprise generating the index value using the formula,

$$f = \frac{1}{1 + {e}^{- Σ {c}_{{i}^{x} i}}}$$

where ‘f’ is the index value, ‘xi’ represents each one of the at least one features, ‘i’ is an integer from 1 to n, where n is the number features, and ‘ci’ represents the reference parameter associated the feature ‘xi’.

The reference parameter may be a weight value applicable to the associated statistical distribution property of the at least one identified feature.

In one embodiment, the GI condition is a functional GI disorder such as irritable bowel syndrome (IBS). The method may comprise determining a likelihood of IBS versus healthy bowels based on the association.

In another embodiment, the GI condition is a GI organic disease such as inflammatory bowel disease (IBD). The method may comprise determining a likelihood of IBD versus healthy bowels based on the association.

In a further embodiment, the GI condition includes IBS and IBD, wherein the at least one statistical distribution property is capable of at least assisting in providing an indication of the existence or non-existence of IBS and IBD.

The method may comprise determining a likelihood of IBS versus IBD based on the association of the at least one statistical distribution property with a corresponding reference parameter.

In one embodiment, the method further comprises simultaneously determining at least one statistical distribution property capable of at least assisting in providing an indication of the existence or non-existence of IBS, and at least one statistical distribution property capable of at least assisting in providing an indication of the existence or non-existence of IBD, whereby the method comprises simultaneously determining a likelihood of IBS versus healthy bowels and a likelihood of IBD versus healthy bowels based on respective associations of the at least one statistical distribution property with corresponding reference parameters.

In one embodiment, the method further comprises determining a likelihood of IBS versus IBD when the respective association of the at least one statistical distribution property with the corresponding reference parameter indicates that IBS is more likely than healthy bowels.

Alternatively, or additionally, the method may further comprise determining a likelihood of IBS versus IBD when the respective association of the at least one statistical distribution property with the corresponding reference parameter indicates that IBD is more likely than healthy bowels.

The at least one feature may comprise, or be based on, one or more of the following: amplitude; burst amount; burst ratio; contraction interval time; higher order zero crossing; band energy ratio; spectral bandwidth; spectral bandwidth double frequency; flatness, spectral centroid; frequency centroid; energy; dynamic range; mel width; envelope crest factor; and roll off.

The method may comprise obtaining the signal representative of the sound including the plurality of bowel sounds using a sound detector. The sound detector may comprise at least two acoustic sensors locatable in proximity to an abdominal region of a subject and spaced-apart from each other for detecting bowel sounds from the abdominal region.

The method may comprise identifying for each bowel sound signal one of the at least two acoustic sensors to be associated with the bowel sound signal based on which sensor produced a highest amplitude reading corresponding to the bowel sound signal.

In order to identify individual bowel sound signals, the method may comprise dividing the signal representative of the bowel sounds into a plurality of segments and, for each segment, determine whether there is a signal portion within any one of the following ranges: 200 Hz to 800 Hz; 600 Hz to 1000 Hz; 800 Hz to 1200 Hz; 1000 Hz to 1600 Hz; and 1600 Hz to 2000 Hz.

The method may comprise identifying a plurality of different features from each of the plurality of bowel sounds signals and determining the likelihood of the GI condition based on a combination of the different features.

In one embodiment, the method comprises determining the likelihood of IBS versus healthy bowels based on a first combination of the different features comprising at least one feature based on: burst; spectral bandwidth double frequency; contraction interval time; or higher order zero crossing.

In another embodiment, the method comprises determining the likelihood of IBD versus healthy bowels based on a second combination of the different features comprising at least one feature based on: flatness; or spectral centroid.

The method may also comprise determining the likelihood of IBS vs IBD based on a third combination of the different features comprising at least one feature based on: envelope crest factor; or roll off.

The method may comprise determining a plurality of different statistical distribution properties of the collection of values for the at least one feature and determine the likelihood of the GI condition based on a combination of the different statistical distribution properties.

According to a third aspect of the invention, there is provided computer readable medium for storing instructions that, when executed by a computing device, causes the computer to perform the method according to the second aspect.

According to a fourth aspect of the invention, there is provided a system for diagnosing a GI condition by analysing bowel sounds, the system comprising:
a sound detector configured to detect bowel sounds and generate a corresponding signal representative of the bowel sounds;
a signal processor arranged to identify a plurality of bowel sound signals within the corresponding signal, each bowel sound signal representative of an individual bowel sound;
wherein the system is arranged to identify at least one feature from each of the plurality of bowel sound signals so as to produce a collection of values for the same at least one feature, and determine at least one statistical distribution property of the collection of values, the statistical distribution property capable of at least assisting in providing an indication of the existence or non-existence of a GI condition; and
wherein the system is further arranged to associate the at least one statistical distribution property with a reference parameter and determine a likelihood of the GI condition based on the association.

According to a fifth aspect of the invention, there is provided a method of diagnosing a GI condition by analysing bowel sounds, the method comprising:
obtaining a signal representative of a sound including a plurality of bowel sounds originating from an abdominal region;
identifying a plurality of bowel sound signals within the signal, each bowel sound signal representative of an individual bowel sound;
identifying at least one feature of each of the plurality of bowel sound signals within the signal so as to produce a collection of values for the same at least one feature;
determining at least one statistical distribution property of the collection of values, the statistical distribution property capable of at least assisting in providing an indication of the existence or non-existence of a GI condition;
associating the at least one statistical distribution property with a reference parameter; and
determining a likelihood of the GI condition based on the association.

### BRIEF DESCRIPTION OF THE DRAWINGS

Notwithstanding any other forms which may fall within the scope of the disclosure as set forth in the Summary, specific embodiments will now be described, by way of example only, with reference to the accompanying drawings in which:

FIG. 1 shows a block diagram of a system according to an embodiment of the invention;

FIG. 2 a block diagram of an identifier of a system according to an embodiment;

FIG. 3 a block diagram of a signal processor of a system according to an embodiment of the invention;

FIG. 4 a block diagram of a determiner of a system according to an embodiment;

FIG. 5 is a top view of showing sensors and a recording device that may be used in a system according to an embodiment;

FIG. 6 is a front view illustrating positions where the sensors shown in FIG. 5 may be located;

FIG. 7 is a representation of a bowel sound signal that may be analysed by the system according to an embodiment;

FIG. 8 shows plots of two signals with particular features;

FIG. 9 shows a block diagram of a system according to a further specific embodiment of the present invention;

FIG. 10 is a flow chart of a method according to an embodiment of the invention; and

FIG. 11 is a flow chart of a process for determining a likelihood of a GI condition in accordance with the further specific embodiment of FIG. 10.

### DETAILED DESCRIPTION OF SPECIFIC EMBODIMENTS

Embodiments of the present invention relate to a method and a system that allow providing a single non-invasive and cost-effective test for indicating a likelihood that a patient may have a gastrointestinal (GI) condition or may have healthy bowels based on the patient's bowel sounds. The GI condition includes a functional GI disorder such as irritable bowel syndrome (IBS), and a GI organic disease such as inflammatory bowel disease (IBD). IBD includes Crohn's disease and ulcerative colitis. It will be understood that embodiments of the invention however may include the determination of a likelihood of functional GI disorder conditions other than IBS, such as cyclic vomiting syndrome functional constipation or functional diarrhea, and may also include the determination of a likelihood of other GI organic diseases, such as coeliac disease, neoplasm, infectious enteritis, obstruction or cancer.

For the diagnosis of IBS, the physician may choose to use the method and system in accordance with embodiments of the present invention after ruling out other diseases, such as IBD, through screening tests or colonoscopy and biopsy. A positive determination or diagnosis of a likelihood of IBS or healthy bowels using the method and system in accordance with embodiments of the present invention would for example allow providing a patient with additional confirmation of a positive IBS diagnosis and that IBD can be ruled out.

The single test using the method and system in accordance with embodiments of the present invention could further allow differentiating, for example, between three groups of patients, namely patients with IBS, patients with IBD and healthy individuals. A physician may then choose to order other tests, such as a colonoscopy with biopsy, to confirm any diagnosis of organic diseases, such as IBD.

### Overview of System

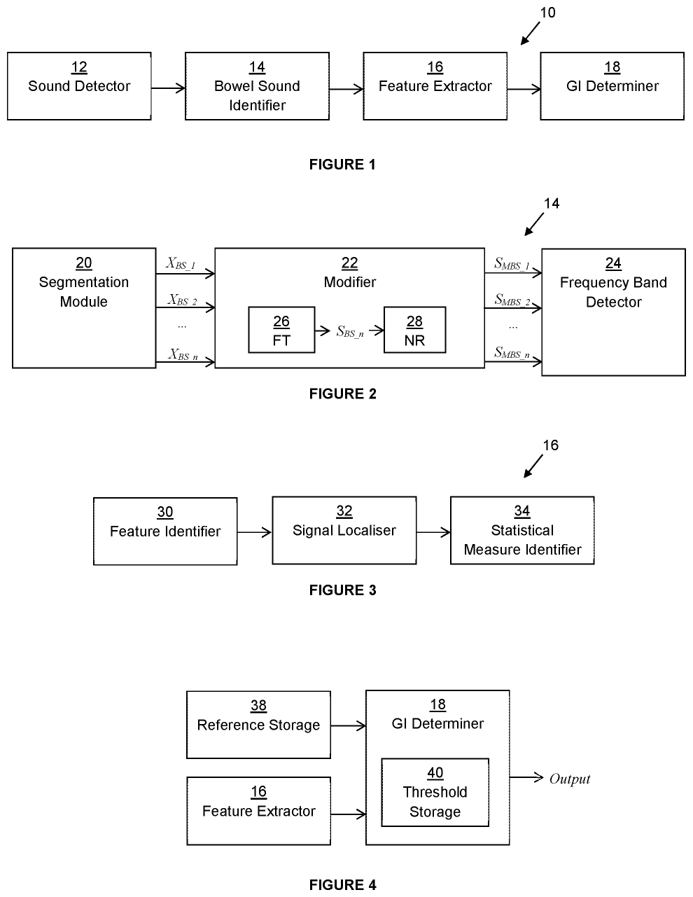

Referring to FIG. 1 of the drawings, an embodiment of a system 10 for indicating a likelihood of a GI condition by analysing bowel sounds is shown. In general terms, the system 10 is configured to obtain a signal corresponding to a continuous recording of bowel sounds, analyse the signal and determine, based on the analysis, the likelihood that the subject producing the bowel sounds has a GI condition.

The system comprises a sound detector 12 for detecting bowel sounds and generating a corresponding signal representative of the bowel sounds. The sound detector 12 can for example be a microphone or piezoelectric sensor. The system 10 also comprises a signal processor arranged to identify a plurality of bowel sound signals within the corresponding signal, wherein each bowel sound signal is representative of an individual bowel sound. In this example, the signal processor comprises a bowel sound identifier 14 for identifying the individual bowel sounds.

The system 10 is arranged to then identify at least one feature from each of the plurality of bowel sound signals so as to produce a collection of values for the same at least one feature. In this example, the signal processor also comprises a feature extractor 16 arranged to extract or identify the at least one feature. The feature(s) may for example be amplitude and/or duration of the bowel sound signals. Preferably, multiple different features are identified from the bowel sound signals, and for each feature a collection of values are obtained. Features that are preferable and/or advantageous will be described in more detail below.

Since a plurality of values for each feature is collected from the plurality of bowel sound signals, a statistical distribution of any one feature can be obtained. The system 10 is then arranged to determine at least one statistical distribution property of the at least one feature, the at least one statistical distribution property being capable of at least assisting in providing an indication of the existence or non-existence of the GI condition. The statistical distribution feature may for example be skewness or kurtosis.

The system 10 is further arranged to associate the at least one statistical distribution property with a reference parameter of a corresponding feature derived using reference data. The system 10 can then determine the likelihood of the GI condition based on the association.

In the example, the system 10 comprises storage for storing the corresponding reference parameters and a GI condition determiner 18 for determining the likelihood based on the association.

In accordance with a first specific embodiment of the invention, the system 10 is configured to determine, based on the association, the likelihood that the subject producing the bowel sounds has IBS as compared to having healthy bowels and the GI condition determiner 18 is an IBS determiner. The system 10 is thus arranged to determine at least one statistical distribution property of the at least one feature, the at least one statistical distribution property being capable of at least assisting in providing an indication of the existence or non-existence of IBS.

In accordance with a second embodiment of the invention, the system 10 is configured to determine, based on the association, the likelihood that the subject producing the bowel sounds has IBD as compared to having healthy bowels and the GI condition determiner 18 is an IBD determiner. The system 10 is thus arranged to determine at least one statistical distribution property of the at least one feature, the at least one statistical distribution property being capable of at least assisting in providing an indication of the existence or non-existence of IBD.

It is noted that patients having Crohn's disease and ulcerative colitis have both been grouped as IBD patients for the purpose of the present invention.

In accordance with a third embodiment of the invention, the system 10 is configured to determine, based on the association, the likelihood that the subject producing the bowel sounds has IBS as compared to having IBD and the GI condition determiner 18 is an IBS/IBD determiner. The system 10 is thus arranged to determine at least one statistical distribution property of the at least one feature, the at least one statistical distribution property being capable of at least assisting in providing an indication of the existence or non-existence of IBS and IBD.

Thus, the system 10 could allow differentiating between healthy individuals and individuals suffering from a functional GI disorder such as IBS, differentiating between healthy individuals and individuals suffering from a GI organic disease such as IBD, and differentiating between individuals suffering from a functional GI disorder such as IBS and individuals suffering from a GI organic disease such as IBD.

In a further embodiment of the invention, the GI condition determiner 18 comprises each of an IBS determiner, an IBD determiner and an IBS/IBD determiner, and the system 10 is configured to determine simultaneously a likelihood of IBS versus healthy bowels and a likelihood of IBD versus healthy bowels based on respective associations of the at least one statistical distribution property with corresponding reference parameters. The system 10 is then further configured to determine a likelihood of IBS versus IBD when the respective association of the at least one statistical distribution property with the corresponding reference parameter indicates that IBS is more likely than healthy bowels. The system 10 is also arranged to determine a likelihood of IBS versus IBD when the respective association of the at least one statistical distribution property with the corresponding reference parameter indicates that IBD is more likely than healthy bowels.

Further, it will be understood that the GI condition determiner 18 may alternatively comprise either one or more of the IBS determiner, IBD determiner and IBS/IBD determiner, and/or other determiners associated with GI conditions other than IBS and IBD.

Components of the system 10 will now be described in more detail.

### Sound Detector

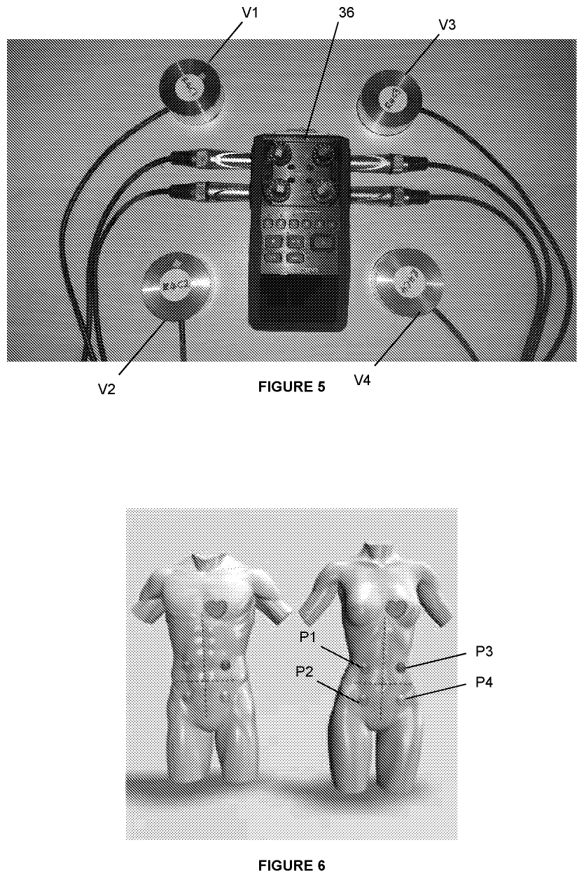

According to a specific example, the sound detector 12 comprises an array of vibration sensors attachable to or held in place by a belt. With reference to FIG. 5, four vibration sensors V1, V2, V3 and V4 according to this example are shown. The vibration sensors V1 to V4 are to be held against a patient's or subject's skin in proximity to an abdominal region in a spaced-apart manner. In this example, the sensors V1, V2, V3 and V4 are placed in positions P1, P2, P3 and P4, respectively, corresponding to quadrants on the subject's abdomen as shown in FIG. 6, using a belt (not shown). The four quadrants included the upper left quadrant (P1), lower left quadrant (P2), upper right quadrant (P3), and lower right quadrant (P4). The belt may be adjustable in length to accommodate for different subjects. For example, the belt may comprise elastic material or Velcro® hook and loop fasteners. Alternatively, the sensors may be held on the subject's skin using an adhesive.

Each vibration sensor V1 to V4 in this example comprises a piezoelectric sensor component and a transducer for converting detected sounds into electrical signals. The sensors V1 to V4 are connected to a recording device for recording the signals, which may also form part of the sound detector 12 component of the system 10. Each vibration sensor could further incorporate double transducers to allow for active noise cancellation if used in a noisy environment. In this example, bowel sounds were recorded in a relatively quiet environment and detected using four single piezoelectric sensors and respective transducers. Piezoelectric sensors are predominantly contact microphones and relatively insensitive to background noise. The recording device may also comprise an analogue-to-digital converter for the purpose of digital signal processing. In this example, hand-held recorder 36 is used, as shown in FIG. 5. However, it will be appreciated that other suitable recording devices may be used.

Preferably, the corresponding signal acquired by the sound detector 12 corresponds to an approximately or over 2-hour recording of bowel sounds from the abdominal region of the subject. In particular, it is proposed that a 2-hour recording of bowel sounds after the subject has fasted for approximately 12 hours, and a further recording of approximately 40 minutes after the subject has had a simple meal (e.g. toast, butter, water, or meal drink such as Sustagen®), may be particularly useful for determining IBS or for determining IBD. Accordingly, the acquired signal may have a signal portion corresponding to a “fasting condition” and another portion corresponding to “food condition”.

### Bowel Sound Identifier

Once the corresponding signal is acquired, the sound detector 12 transmits the signal to the bowel sound identifier 14. The signal transmission may be via wireless or wired data transmission means. The bowel sound identifier 14 then processes the signal to automatically identify individual bowel sound signals. With reference to FIG. 2, the identifier 14 comprises a segmentation module 20, a signal modifier 22 and a frequency band detector 24.

The segmentation module 20 divides the corresponding signal into signal segments, X_{BS_1}, X_{BS_2}, . . . , X_{BS_N}, where X_{BS} is bowel sound time series data. The segments may for example be 20-40 ms in length, such as approximately 30 ms in length. In this specific example, the segmentation module 20 utilises a window function, which may have a window size of approximately 30 ms, with 20 ms overlap between adjacent windows. As bowel sounds are usually short bursts, where the energy versus time distribution is extremely uneven, a rectangular window function was selected.

The signal modifier 22 then applies a Fourier transformation 26 to the signal segments to obtain frequency spectrum data, S_{BS_1}, S_{BS_2}, . . . , S_{BS_N}, as follows:

S_{BS}=FFT(X_{BS}) (Eq. 1)

The frequency response (S_{N}) of the sound detector 12 is also evaluated for the purpose of removing background noise from the spectrum, as follows:

S_{N}=FFT(X_{Noise}) (Eq. 2)

The modifier 22 then performs noise reduction 28 to obtain a series of modified spectrum, S_{MBS_1}, S_{MBS_2}, . . . , S_{MBS_N}, corresponding to each signal segment, as follows:

S_{MBS}=S_{BS}/S_{Noise} (Eq. 3)

The series of modified spectrum data S_{MBS_1}, S_{MBS_2}, . . . , S_{MBS_N}, is then inputted into a frequency band detector 24 of the bowel sound identifier 14 in order to identify or designate individual bowel sound signals. Alternatively or additionally, active noise cancellation could be performed prior to identification of bowel sounds.

In this regard, it was recognised that the main frequency component of all of the bowel sounds was between 200 and 2000 Hz with a relatively narrow bandwidth. In contrast, other contaminating noise factors may be present in the spectrum data, such as friction against the sensors, lung sounds, and the heart beating, etc. However, it was determined that the frequency spectra of these noises did not overlap with the bowel sounds. Thus, in order to automatically identify bowel sounds, a plurality of specific frequency band subsets are selected. In this example, the following five frequency bands were used:
1. 200 to 800 Hz
2. 600 to 1000 Hz
3. 800 to 1200 Hz
4. 1000 to 1600 Hz
5. 1600 to 2000 Hz

The term band energy ratio (BER) is used herein to mean the ratio of energy that a particular signal or signal portion has within a specific frequency band over the full range of frequencies present in the recording. For each signal segment, a frequency band detector 24 calculates the BER that a signal segment has within specific frequency bands. If the detector 24 identifies that a signal segment has a BER higher than a threshold, such as 90%, within one of the frequency bands, the signal segment is recognised as a bowel sound section. Further, if the detector 24 recognises no other bowel sound section within a range of 100 ms either side of the recognised bowel sound section, the detector 24 defines the bowel sound section as an individual bowel sound signal. Alternatively, if more than one bowel sound section was recognised within the time frame of the signal segment, the detector 24 groups the bowel sound sections and defines the grouping as a single bowel sound signal with multiple components.

In an over 2-hour long recording of bowel sounds, it is estimated that there may be hundreds of thousands of individual bowel sound signals identified.

The system 10 then inputs the identified bowel sound signals into the feature extractor 16.

### Feature Extractor

In this example, the system 10 comprises a feature extractor 16 arranged to identify a plurality of different features from each of the plurality of bowel sounds signals. The different features may for example be features based on burst (such as burst amount or burst ratio), contraction interval time, spectral bandwidth double frequency, band energy ratio, higher order zero crossing, flatness, spectral centroid, energy, dynamic range, mel width, envelope crest factor, or roll off, which will be described in more detail below. In this example, based on a combination of the different features, the GI condition determiner 18 is arranged to determine the likelihood of the GI condition.

In the first embodiment, the IBS determiner of the GI condition determiner 18 is arranged to determine a likelihood of IBS versus healthy bowels based on a first combination of the different features comprising at least one feature based on burst, spectral bandwidth double frequency, contraction interval time, or higher order zero crossing.

In the second embodiment, the IBD determiner of the GI condition determiner 18 is arranged to determine a likelihood of IBD versus healthy bowels based on a second combination of the different features comprising at least one feature based on flatness or spectral centroid.

In a third embodiment, the IBS/IBD determiner of the GI condition determiner 18 is arranged to determine a likelihood of IBS versus IBD based on a third combination of the different features comprising at least one feature based on envelope crest factor or roll off.

Before describing the feature extractor 16 in detail, a specific process that was conducted for selecting preferable features to extract from the bowel sound signals will be described. However, it will be understood that embodiments of the invention are not limited thereto, and variations on the process described herein may be utilised to selected features.

### Feature Selection

Using the sound detector 12 described above, an experiment was conducted whereby 2-hour long bowel sound recordings were obtained from participants after fasting, and a further 40 minutes recording was obtained after the participants had a standard meal. Hence, 160 minute recordings were obtained from each participant using the four sensors V1 to V4. The positioning of the sensors as shown in FIG. 6 allowed for the gathering of information on gastrointestinal activities from the stomach through to the small and large intestines, where IBS is known to alter motility, and where IBD is known to affect the structure of the intestines, which will also affect sound generation.

The recordings were sampled at a sampling rate of 44.1 kHz, equating to approximately 1.6 billion samples. Signal processing was performed in order to reduce the number of samples and extract features from the samples. In particular, the bowel sound identification process described above as being performed by the bowel sound identifier 14 was conducted, resulting in a collection of individual bowel sound signals and respective frequency spectrum data for each participant.

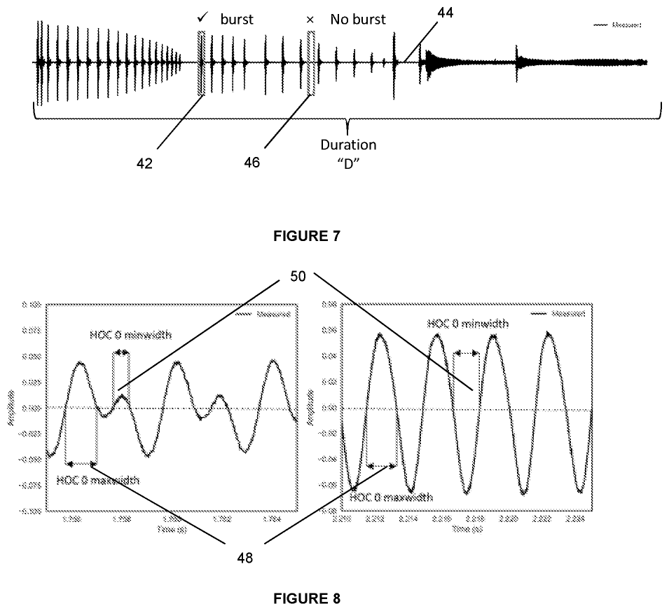

Initially, several time domain features and frequency domain features were identified for each individual bowel sound signal. The time domain features include the following.
(a) Burst: The number of bowel sound sections (i.e. the bowel sound sections identified by the Bowel Sound Identifier) within a bowel sound signal. FIG. 7 illustrates a section 42 of a bowel sound signal 44 that constitutes a “burst”, as well as a section 46 of the signal 44 that does not constitute a burst.
(b) Duration: The duration of a bowel sound signal. For example, FIG. 7 illustrates duration “D” of an individual bowel sound signal.
(c) Burst Ratio: The duration over the burst, which can be determined using Equation 4 below.

BR=Duration/Burst (Eq. 4)
(d) Contraction Interval Time (CIT): The time interval (Tₖ) between subsequent muscle contractions, which can be determined by considering a model for a complete bowel sound provided by Equation 5 below.

$${S}_{b} = {\sum }_{i = 1}^{Burst} \frac{{PI}_{i} sin ( 2 π {f}_{iwc} ( t - {\sum }_{k = 1}^{l} {T}_{k} ) )}{{( t - {\sum }_{k = 1}^{i} {T}_{k} )}^{b} ( exp ( \frac{E}{t - {f}_{iwc} {\sum }_{k = 1}^{i} {T}_{k}} ) - 1 )} + n ( t ) \qquad ( Eq . 5 )$$

(e) Dynamic Range: The maximum peak-to-peak value of the bowel sound signal.

DR=20*log₁₀(max(X_{BS})−min(X_{BS}))) (Eq. 6)
(f) Amplitude: The maximum value within an individual bowel sound, which can be determined using Equation 7 below.

A=max(X_{BS}) (Eq. 7)
(g) Energy (En): The energy of the bowel sound.

En=20*log₁₀(mean(X_{BS}²)) (Eq. 8)
(h) Envelope Crest Factor (ECF): The ratio of the peak value to the mean value to show how extreme the peaks are in a waveform.
(i) Higher order zero crossing (HOCₙ): The ratio between the time the waveform changes its sign and the total data quantity, i.e. the quantity of sample points within the bowel sound. The mean, minimum and the maximum value of the width between the adjacent crossing points are extracted as features. For example, FIG. 8 shows a maximum HOCₙ (item 48) and a minimum HOCₙ (item 50) in two different graphs.

The mean of all HOCₙ values identified from the plurality of bowel sound signals for a participant can be determined by the Equation 7, as follows.

$$HOCn = mean ( \frac{{\partial }^{n} {X}_{BS} ( 1 : N - 1 )}{\partial {t}^{n}} * \frac{{\partial }^{n} {X}_{BS} ( 2 : N )}{\partial {t}^{n}} < 0 ) \qquad ( Eq . 9 )$$

where n equals to 0, 1, 2 or 3 to represent different order of differentiation.
(j) and (k) End and Start: the time stamps of the bowel sound.

The frequency domain features include the following.
(a) Spectral Centroid: Denoting the centre of the spectrum, the powers of the amplitude are 1 and 2, respectively.
(b) Band Energy Ratio (HBER): The energy ratio of a particular frequency band over the whole frequency, which can be determined by Equation 9 below.

$$HBERk\_i \_j = \frac{{\sum }_{i}^{j} {S}_{MBS}^{k}}{\sum {S}_{MBS}^{k}} \qquad ( Eq . 10 )$$

where k equals to 1 or 2 to represent different calculating order.
(c) Frequency Centroid (FC)

$$FCj = \frac{{Σ}_{n = 1}^{N} ( {S}_{MBS , i}^{j} ( n ) * f ( n ) )}{{Σ}_{n = 1}^{N} {S}_{MBS , i}^{j} ( n )} \qquad ( Eq . 11 )$$

where j equals to 1 or 2 to represent different calculating order.
(d) Spectral Band Width: The wavelength interval in which a radiated spectral quantity is not less than half its maximum value. SBW1 and SBW2 are two different types of the band width according to Equation 11,

$$SBWj = \frac{{Σ}_{n = 1}^{N} ( {S}_{MBS , i}^{j} ( n ) * {( f ( n ) - F C )}^{2} )}{{Σ}_{n = 1}^{N} {S}_{MBS , i}^{j} ( n )} \qquad ( Eq . 12 )$$

(e) Modified Spectral Band Width: The spectral band width but with a modification to include a power of 2, as shown in Equation 12 below, using Equation 10 to determine the frequency centroid (FC), i.e. a centre of the frequency.

$$SBWDFj = \frac{{{Σ}_{n = 1}^{N} ( {S}_{MBS , i}^{j} ( n ) * ( f ( n ) - F C ) )}^{2}}{{Σ}_{n = 1}^{N} {S}_{MBS , i}^{j} ( n )} \qquad ( Eq . 13 )$$

(f) Spectral Flatness: A measure to indicate the flatness of the frequency spectrum of a bowel sound signal.
(g) Highest Energy Frequency (HEF): The frequency with highest amplitude component.
(h) Spectrum Skewness: The skewness of the spectrum.
(i) Spectrum Kurtosis: The kurtosis of the spectrum.
(j) Sub-Band Contrast: A feature for describing the timbre using the amplitude ratio between the frequencies with the highest values (e.g. top 20%) and the lowest values (e.g. bottom 20%).
(k) Mel-Frequency: The maximum band (MelMax), band number over certain value (MeWdith), and the sum of the band on the 13 band Mel-frequency (MelSum). The Mel-frequency band number can be determined using Equation 13 below,

$${S}_{M e l} = 2595 {log}_{10} ( 1 + \frac{{S}_{M B S}}{700} ) \qquad ( Eq . 14 )$$

(l) MelMax and Mel-sum can be determined using Equations 14 and 15 below.

MelMax=max(S_{Mel}) (Eq. 15)

MelSum=ΣS_{Mel} (Eq. 16)
(m) MelWidth: The number of the band whose energy larger than 10% of the maximum energy in Mel scale (Mel scale in Eq. 11)

MelWidth=(S_Mel>max(S_Mel)) (Eq. 17)
(n) Rolloff: This is a measure of the amount of the right-skewedness of the power spectrum. This feature is defined as the frequency under which 95% of the signal's spectral energy is accumulated. N is the number of spectrum data points S,

$$Rolloff = \frac{len ( S > 0.05 * {S}_{m ax} )}{N} \qquad ( Eq . 18 )$$

(o) Flatness 3000: Spectral Flatness, is a measure used in digital signal processing to characterize an audio spectrum,

$${Flatness}_{j , 3000} = \frac{{e}^{\frac{1}{N} \sum l n ( {S}^{j} ( n ) )}}{\frac{1}{N} \sum {S}^{j} ( n )} \\ S ( n ) ∈ [ 1 , 3000 ] \qquad ( Eq . 19 )$$

where j equals to 1 or 2 to represent different calculating order.

The features above were extracted from each individual bowel sound signal or respective spectrum to construct a bowel sound library.

Additionally, to characterise the bowel activity further, the individual bowel sounds signals were assigned to one of the four sensors V1 to V4 positioned at different abdominal quadrants P1 to P4. The assigning of individual bowel sound signals to a particular abdominal quadrant was done according to the amplitude of the bowel sound signal and on the assumption that the sensitivity of each the sensors were identical. In particular, bowel sound signals that were detected by multiple sensors V1 to V4 were assigned to the quadrant/sensor that was most strongly associated with the bowel sound signal. In this example, each bowel sound signal was associated with the sensor V1 to V4 that produced the highest amplitude reading for that bowel sound. For instance, if the same bowel sound was detected by two sensors, the sensor that registered the corresponding bowel sound signal with the highest amplitude reading would be selected. Accordingly, each bowel sound signal would only be associated with one sensor/quadrant. Additionally, a minimum threshold of 60% of maximum energy was applied. Therefore, if for example a bowel sound originated from a relatively central region, it would only be assigned to a quadrant if that quadrant obtained a reading of the bowel sound that exceeded the threshold.

Since hundreds of thousands of bowel sound signals were identified from each participant, a corresponding amount of values for each feature can be extracted, and the statistical distribution of each feature can be statistically analysed. It was found that the statistical distribution of the features was different in participants with IBS compared to healthy participants, and also that the statistical distribution of the features was different in participants with IBD compared to healthy participants. Further, the statistical distribution of the features was different in participants with IBD compared to participants with IBS. In all three cases, for some features the difference was to a greater degree than others. This was evident by examining the skewness and kurtosis of the statistical distribution of the features. In other words, the skewness and kurtosis of the distribution of features contributed significantly to classification of participants. A reason for this could be that there is greater variability in the distribution of sounds from IBS participants given their altered motility pattern, and from IBD participants given the underlying motility and structural changes

As a result, a collection of “hybrid” features was obtained for the bowel sound signals, the hybrid features having several components including: (a) feature, e.g. amplitude, burst; (b) statistical distribution feature, e.g. skew, kurtosis; (c) assigned sensor; and (d) a condition, e.g. fasting or food.

Logistic regression analysis was then used to identify the optimal array or subset of all the hybrid features (taking into account associated quadrants P1 to P4) that was most strongly associated with participants having a GI condition, i.e. in the present examples IBS or IBD, and healthy participants. The logistic regression analysis firstly uses a linear regression model and then a sigmoid function to predict the probability of a sample being positive. No assumption about the data distribution are made when using logistic regression, but the correlation coefficients among each of the features should be smaller than 0.7 to obtain a stable and reasonable result. The particular linear regression model and sigmoid functions used are provided in Equations 20 and 21, respectively.

$$\begin{matrix}{\sum }_{n}^{i} {c}_{i} {x}_{i} = {c}_{1} {x}_{1} + {c}_{2} {x}_{2} … + {c}_{n} {x}_{m} & ( Eq . 20 ) \\ f = \frac{1}{1 + {e}^{- \sum {c}_{i} {x}_{i}}} & ( Eq . 21 )\end{matrix}$$

In Equations 20 and 21 above, ‘xᵢ’ represents one of the features, where ‘i’ is an integer from 1 to n, with n being the total number of features, and ‘c’ is a weighting coefficient associated with each one of the features ‘xᵢ’.

In the first embodiment wherein, the system 10 is arranged to determine a likelihood of IBS versus healthy bowels, the weighting coefficients were then adjusted using a cost function so that f=1 if a bowel sound belongs to a participant having IBS, and f=0 to indicate that the participant does not have IBS. Use of the cost function allows for determination of respective weighting coefficients for the various features ‘xᵢ’, which would minimise the “cost” that a particular coefficient would have on the result, thus optimising the result, i.e. towards f=1 for a bowel sound associated with IBS and towards f=0 for a bowel sound that is not indicative of IBS. The weighting coefficients were first assigned a random number, and then adjusted to conform to the participants true condition, whether IBS or healthy. This was repeated multiple times until the accuracy of the coefficients stopped improving.

In the second embodiment, the same iterative process and background logistic regression model, including the linear regression model (Eq. 20), sigmoid function (Eq. 21) and assumptions, were used to identify the optimal array or subset of all the hybrid features (taking into account associated quadrants P1 to P4) that was most strongly associated with IBD participants and healthy participants. In this case, the weighting coefficients were then adjusted using a cost function so that f=1 if a bowel sound belongs to a participant with IBD, and f=0 to indicate that the participant does not have IBD. The weighting coefficients were first assigned a random number, and then adjusted to conform to the participants true condition, whether IBD or healthy. This was repeated multiple times until the accuracy of the coefficients stopped improving.

The same method was also used in the third embodiment for differentiation between IBD and IBS individuals. The same iterative process and background logistic regression model, including the linear regression model (Eq. 20), sigmoid function (Eq. 21) and assumptions, were used to identify the optimal array or subset of all the hybrid features (taking into account associated quadrants P1 to P4) that was most strongly associated with IBD participants and IBS participants. In this case the weighting coefficients were then adjusted using a cost function so that f=1 if a bowel sound belongs to a participant with IBD, and f=0 to indicate that the participant does not have IBS.

Along with the logistic regression, regularisation was used for preventing over-fitting. There are two common regularisation methods: L1 and L2. The latter was selected for its potential to increase the generalisation of the model by reducing the absolute value of the weights and thus prevent perfect fitting.

In order to corroborate the accuracy of the model, cross-validation was also performed. In particular, the leave-one-out cross validation (LOOCV) method was used for tuning the parameters and selecting features. The LOOCV procedure involves removing one sample and training the model using the rest of the samples, before calculating the error on the sample which was removed. Alternatively or additionally, bootstrapping is another method for cross-validating the selected features and model.

As there are thousands of hybrid features that can be used in the model, all skewness-related features were initially included as a starting point in the process of determining an optimal subset of features. Each feature was then removed one by one and the LOOCV performance of the remaining features was analysed. The features subset with highest LOOCV accuracy was retained before the remaining features was removed one by one. This process was repeated until accuracy plateaued. Subsequently, additional features from the whole features set were added to the model one by one until the maximum accuracy was achieved.

In addition, in the third embodiment in particular wherein the system 10 is arranged for determining a likelihood of IBS versus IBD, the logistic regression analysis can be affected by a problem of imbalanced data set, i.e. a difference between the number of sample participants having IBS and the number of sample participants having IBD, which may create a bias towards IBD. Indeed, the sample size of IBD is typically much greater than the sample size of IBS. In order to correct for this bias and corroborate the accuracy of the model for differentiating between IBS and IBD in particular, a method of oversampling was used to increase the sample size of IBS and healthy recordings to match the number of IBD recordings. The oversampling method involved generating new samples using the following equation:

x_{(new)}=xᵢ+μ*(x_{{zi}}−xᵢ) (Eq. 22)

where μ is a random number in the range [0,1], and where this interpolation creates a sample on the line between xᵢ and x_{{zi}}, x_{{zi}} being nearest-neighbours to xᵢ.

In the first embodiment of differentiation between IBS and healthy bowels, a total of 26 optimal or “ultimate” features were identified from among the hybrid features to form part of the optimum model. These features are provided in Table 1 below together with an example of respective weighting coefficients for those features. As shown below,

TABLE 1

| No. | Ultimate Feature | Coefficient |
|---|---|---|
| 1 | Amplitude_kurtosis_Ch3_x | +0.014 |
| 2 | BurstRatio_kurtosis_Ch1_x | −0.560 |
| 3 | Burst_kurtosis_Ch1_x | −0.008 |
| 4 | Burst_skew_Ch3_y | +0.490 |
| 5 | CIT_skew_Ch1_y | −0.394 |
| 6 | HOC0_minwidth_skew_Ch2_x | +0.084 |
| 7 | HOC0_minwidth_skew_Ch3_y | +0.322 |
| 8 | HOC2_skew_Ch1_y | +0.368 |
| 9 | HBER2_1000_1500_kurtosis_Ch1_x | +0.145 |
| 10 | HBER2_2500_5000_skew_Ch1_x | +0.117 |
| 11 | HBER2_2500_5000_skew_Ch1_y | −0.248 |
| 12 | HBER2_2500_5000_skew_Ch2_x | −0.161 |
| 13 | MelMax_kurtosis_Ch3_y | +0.411 |
| 14 | MelMax_skew_Ch4_y | +0.564 |
| 15 | MelSum_50Q_Ch2_x | +0.045 |
| 16 | MelSum_50Q_Ch4_y | −0.031 |
| 17 | SBWDF1_kurtosis_Ch1_y | −0.013 |
| 18 | SBWDF1_skew_Ch1_y | +0.368 |
| 19 | SBWDF2_skew_Ch1_y | +0.095 |
| 20 | SBWDF2_skew_Ch2_x | +0.354 |
| 21 | SBWDF2_skew_Ch3_x | −0.336 |
| 22 | SBWDF2_skew_Ch4_x | −0.309 |
| 23 | SpectrumKurtosis_kurtosis_Ch1_y | −0.006 |
| 24 | SpectrumKurtosis_skew_Ch3_y | −0.350 |
| 25 | SpectrumKurtosis_skew_Ch4_x | +0.302 |
| 26 | SpectrumSkew_skew_Ch3_x | −0.728 |

In the embodiment of differentiation between IBD and healthy bowels, a total of 44 optimal or “ultimate” features were identified from among the hybrid features to form part of the optimum model. These features are provided in Table 2 below together with an example of respective weighting coefficients for those features. As shown below,

TABLE 2

| No. | Ultimate Feature | Coefficient |
|---|---|---|
| 1 | Amplitude_skew_Ch1_x | −0.0241 |
| 2 | BurstRatio_skew_Ch1_x | 0.0463 |
| 3 | CIT_kurtosis_Ch3_y | −0.0072 |
| 4 | CIT_kurtosis_Ch4_x | 0.0098 |
| 5 | DR_skew_Ch1_x | 0.0135 |
| 6 | ECF_skew_Ch2_x | 0.0570 |
| 7 | En_kurtosis_Ch3_y | −0.0068 |
| 8 | En_skew_Ch1_x | 0.0139 |
| 9 | End_kurtosis_Ch3_y | −0.0002 |
| 10 | FCC1_kurtosis_Ch3_y | 0.0397 |
| 11 | FCC1_skew_Ch1_x | −0.0064 |
| 12 | FCC2_skew_Ch_1_x | 0.0261 |
| 13 | Flatness_1_3000_kurtosis_Ch2_y | 0.0370 |
| 14 | Flatness_1_3000_skew_Ch1_x | 0.0072 |
| 15 | Flatness_2_3000_kurtosis_Ch2_y | 0.1036 |
| 16 | HBER_1_2500_5000_kurtosis_Ch1_y | 0.0163 |
| 17 | HBER_1_2500_5000_skew_Ch3_x | 0.1273 |
| 18 | HBER_2_1000_1500_kurtosis_Ch2_x | 0.0092 |
| 19 | HBER_2_1000_1500_kurtosis_Ch4_x | −0.0041 |
| 20 | HBER_2_200_500_skew_Ch1_x | −0.0081 |
| 21 | HBER_2_2500_5000_kurtosis_Ch1_x | −0.0005 |
| 22 | HBER_2_2500_5000_kurtosis_Ch1_x | −0.0005 |
| 23 | HBER_2_500_800_skew_Ch1_x | 0.0229 |
| 24 | HBER_2_800_1000_kurtosis_Ch4_y | 0.0224 |
| 25 | HEF_skew_Ch1_x | 0.0313 |
| 28 | HOC0stdwidth_skew_Ch1_x | 0.0082 |
| 29 | HOC1_skew_Ch1_x | −0.0126 |
| 30 | HOC2_skew_Ch1_x | −0.1153 |
| 31 | HOC3_kurtosis_Ch4_y | −0.0407 |
| 32 | MelMax_skew_Ch1_x | 0.0433 |
| 33 | MelSum_kurtosis_Ch4_x | −0.0027 |
| 34 | MelWidth_kurtosis_Ch2_y | 0.1281 |
| 35 | MelWidth_skew_Ch1_x | −0.0244 |
| 36 | SBW1_skew_Ch1_x | 0.0013 |
| 37 | SBWDF1_kurtosis_Ch2_y | 0.001 |
| 38 | SBWDF1_kurtosis_Ch4_x | −0.0055 |
| 39 | SBWDF1_skew_Ch1_y | −0.0074 |
| 40 | SBWDF2_kurtosis_Ch1_x | −0.0011 |
| 41 | SBWDF2_kurtosis_Ch2_x | 0.0003 |
| 42 | SBWDF2_kurtosis_Ch4_x | −0.0001 |
| 43 | SpectrumSkew_skew_Ch1_x | −0.0072 |
| 44 | Start_skew_Ch1_x | −0.0076 |

In the embodiment of differentiation between IBD and IBS, a total of 26 optimal or “ultimate” features were identified from among the hybrid features to form part of the optimum model. These features are provided in Table 3 below together with an example of respective weighting coefficients for those features. As shown below,

TABLE 3

| No. | Ultimate Feature | Coefficient |
|---|---|---|
| 1 | Amplitude_kurtosis_Ch4_x | −0.0545 |
| 2 | Burst_kurtosis_Ch2_{——}y | −0.0681 |
| 3 | CIT_kurtosis_Ch1_x | 0.0059 |
| 4 | CIT_kurtosis_Ch4_y | −0.0206 |
| 5 | Duration_kurtosis_Ch1_x | −0.0445 |
| 6 | Duration_kurtosis_Ch3_x | 0.0317 |
| 7 | ECF_kurtosis_Ch2_y | −0.0745 |
| 8 | HBER2_1000_1500_kurtosis_Ch1_y | 0.0338 |
| 9 | HBER2_1500_2500_kurtosis_Ch2_x | 0.1726 |
| 10 | HBER2_1500_2500_kurtosis_Ch3_x | −0.0836 |
| 11 | HBER2_2500_5000_kurtosis_Ch2_x | −0.0042 |
| 12 | HBER2_2500_5000_kurtosis_Ch2_y | 0.0193 |
| 13 | HBER2_2500_5000_kurtosis_Ch3_y | 0.0056 |
| 14 | HBER2_2500_5000_kurtosis_Ch4_y | 0.0036 |
| 15 | HBER2_800_1000_kurtosis_Ch4_x | −0.0301 |
| 16 | HBER2_800_1000_kurtosis_Ch4_y | 0.0691 |
| 17 | HOC0minwidth_kurtosis_Ch2_y | −0.0097 |
| 18 | HOC3_kurtosis_Ch4_x | 0.064 |
| 19 | RollOff_1_kurtosis_Ch4_x | −0.0134 |
| 20 | SBW2_kurtosis_Ch2_x | 0.0668 |
| 21 | SBWDF1_kurtosis_Ch1_x | −0.0252 |
| 22 | SBWDF2_kurtosis_Ch1_x | 0.0161 |
| 23 | SBWDF2_kurtosis_Ch2_y | 0.0236 |
| 24 | SpectrumKurtosis_kurtosis_Ch1_y | −0.0541 |
| 25 | SpectrumKurtosis_kurtosis_Ch4_x | 0.0219 |
| 26 | SpectrumKurtosis_kurtosis_Ch4_y | −0.0121 |

It will be appreciated that the features and respective weighting coefficients above are examples only, and in other embodiments different features and values for weighting coefficients may be used. As mentioned above, in the listing of ultimate features, four components are represented in each feature, where a first component corresponds to a “feature”, a second to a“statistical measure”, a third to a“sensor” and a fourth to a “condition”. Each component is separated by an underscore and is selected among the following listed in Table 4 below.

TABLE 4

| | Statistical | | |
|---|---|---|---|
| Feature | Measure | Sensor | Condition |
| Amplitude | Kurtosis | Ch1 = | x = |
| Burst Ratio | Skew | V1 | fasting |
| Burst | Median | Ch2 = | y = |
| CIT | | V2 | food |
| Duration | | Ch3 = | |
| Dynamic Range (DR) | | V3 | |
| ECF | | Ch4 = | |
| Energy | | V4 | |
| FCC1 | | | |
| FCC2 | | | |
| Flatness_1_3000 | | | |
| Flatness_2_3000 | | | |
| HBER1 (2500_5000) | | | |
| HBER2 (200_500, | | | |
| 500_800, 800_1000, | | | |
| 1000_1500, 1500_2500, | | | |
| 2500_5000) | | | |
| HEF | | | |
| HOC0_minwidth | | | |
| HOC0_maxwidth | | | |
| HOC0_stdwidth | | | |
| HOC1 | | | |
| HOC2 | | | |
| HOC3 | | | |
| MelMax | | | |
| MelSum | | | |
| MelWidth | | | |
| Roll-off | | | |
| SBW1 | | | |
| SBW2 | | | |
| SBWDF1 | | | |
| SBWDF2 | | | |
| Spectrum Kurtosis | | | |
| Spectrum Skew | | | |
| Start | | | |

Although a specific experiment has been described above to obtain the respective 26 or 44 ultimate features to be used in the system 10, those skilled in the art will understand that other methods of obtaining desirable features, and other combinations of features, may be selected according to other embodiments.

### Feature Extraction

Continuing with the embodiment shown in FIG. 1, once the bowel sound identifier 14 has identified the plurality of individual bowel sound signals from a sound recording, the bowel sound signals (or corresponding frequency spectrums) are inputted into the feature extractor 16.

For each bowel sound signal, the feature extractor 16 identifies selected features from each of the plurality of bowel sound signals so as to produce a collection of values for each of the selected features. In the present examples, the selected features are the 26 ultimate features listed above in Table 1 when the system 10 is arranged for determining a likelihood of IBS versus healthy bowels, the 44 ultimate features identified in Table 2 when the system 10 is arranged for determining a likelihood of IBD versus healthy bowels, or the 26 features identified in Table 3 when the system 10 is arranged for determining a likelihood of IBS versus IBD. The feature extractor 16 then determines at least one statistical distribution property of the collection of values.

The feature extractor 16 comprises a feature identifier 30, a signal localiser 32 and a statistical measure identifier 34.

In the example of differentiation between IBS and healthy individuals, the feature identifier 30 is configured to extract the features listed in Table 1 above (column 1) from the bowel sound signals received from the bowel sound identifier 14. For example, the feature identifier 30 may extract the CIT feature from each bowel sound signal by utilising Equation 5 above.

In the example of differentiation between IBD and healthy individuals, the feature identifier 30 is configured to extract the features listed in Table 2 above (column 1) from the bowel sound signals received from the bowel sound identifier 14. For example, the feature identifier 30 may extract the flatness 3000 feature from each bowel sound signal by utilising Equation 19 above.

In the example of differentiation between IBS and IBD individuals, the feature identifier 30 is configured to extract the features listed in Table 3 above (column 1) from the bowel sound signals received from the bowel sound identifier 14. For example, the feature identifier 30 may extract from each bowel sound signal the envelope crest factor feature and/or the roll off feature by utilising Equation 18 above.

Since a plurality of bowel sound signals are identified for each subject, the feature identifier 30 will then output a collection or series of values for each feature. As an example only, for each bowel sound recording the following collection of features may be obtained for amplitude and burst:

TABLE 5

| Feature | Collection of Values |
|---|---|
| Amplitude | A1 = max (X_{BS}_{_{—}}₁) = 0.5 |
| | A2 = max (X_{BS}_{_{—}}₂) = 0.6 |
| | A3 = max (X_{BS}_{_{—}}₃) = 0.8 |
| | etc. |
| Burst | burst (X_{BS}_{_{—}}₁) = 12 |
| | burst (X_{BS}_{_{—}}₂) = 18 |
| | burst (X_{BS}_{_{—}}₃) = 10 |
| | etc. |

The signal localiser 32 is configured to then assign each bowel sound signal to one of the sensors V1 to V4. As described above in relation to the “Feature Selection”, the assigning of bowel sound signals was done by assigning the signal to the sensor V1 to V4 that detected the highest amplitude, while applying a minimum threshold of 60% of the maximum energy. As an example only, signal localiser 32 may obtain the following:

TABLE 6

| Signal | Dominant Sensor |
|---|---|
| X_{BS}_{_{—}}₁ | V1 |
| X_{BS}_{_{—}}₂ | V4 |
| X_{BS}_{_{—}}₃ | V2 |

The statistical measure identifier 34 is arranged to then determine a plurality of different statistical distribution properties of the collection of values for the features. In particular, with reference to Tables 1, 2 and 3 above, the statistical distribution properties calculated include kurtosis and skewness of the collection of values for specific features and specific sensors. For example, with reference to the 26 features in Table 1, the identifier 34 would calculate values for the kurtosis of the collection of amplitude values of signals assigned to V3 (feature no. 1), and the skew of the collection of burst values of signals assigned to V3 (feature no. 4). Additionally, the statistical measure identifier 34 also calculates the median of the sum of Mel-frequencies of signals assigned to V2 (feature no. 15).

The statistical measure identifier 34 in this example uses the following equations to identify skewness and kurtosis:

$$\begin{matrix}{F}_{skew} = \frac{\frac{1}{{N}_{BS}} \sum {( F - \frac{1}{{N}_{BS}} \sum F )}^{3}}{{( \frac{1}{{N}_{BS}} \sum {( F - \frac{1}{{N}_{BS}} \sum F )}^{2} )}^{3 / 2}} & ( Eq . 23 ) \\ {F}_{kurtosis} = \frac{\frac{1}{{N}_{BS}} \sum {( F - \frac{1}{{N}_{BS}} \sum F )}^{4}}{{( \frac{1}{{N}_{BS}} \sum {( F - \frac{1}{{N}_{BS}} \sum F )}^{2} )}^{2}} & ( Eq . 24 )\end{matrix}$$

In Equations 23 and 24 above, the variable “F” is a value of the feature being examined such that the sum of all the values of the feature is evaluated in the equations above, and the variable “N_{BS}” represents the number of bowel sounds. Values for the 26 selected features in Table 1 above are thus obtained from the recorded bowel sound.

Similarly, in the example with reference to the 44 features in Table 2, the identifier 34 would calculate values for the kurtosis of the collection of flatness 3000 values of signals assigned to V2 (features no. 13 and 15), and the skew of the collection of spectral centroid values of signals assigned to V1 (features no. 11 and 12). The statistical measure identifier 34 in this example uses the equations 23 and 24 to identify skew and kurtosis and values for the 44 selected features in Table 2 above are thus obtained from the recorded bowel sound.

In the third example to differentiate between IBD and IBS individuals, the same method could be employed. In the example with reference to the 26 features in Table 3, the identifier 34 would calculate values for the kurtosis of the collection of envelope crest factor values of signals assigned to V2 (feature no. 7) and the kurtosis of the collection of roll off values of signals assigned to V4 (feature no. 19). The statistical measure identifier 34 in this example uses the equations 23 and 24 to identify skew and kurtosis and values for the 26 selected features in Table 3 above are thus obtained from the recorded bowel sound.

### Determiner

In this example, the system 10 comprises the GI condition determiner 18 for determining the likelihood that the subject from which the bowel sounds are obtained has the respective GI condition versus having healthy bowels, and preferably outputs an index value indicative of that likelihood. The determiner 18 communicates with reference storage 38 and the feature extractor 16. The reference storage 38 stores reference parameters associated with each of the ultimate features. The reference parameters may for example be a coefficient, constant value, variable or property.

In the example when determining a likelihood of IBS versus healthy bowels, the reference parameters are the weighting coefficients listed in Table 1 above, which were derived from the process of selecting the optimum hybrid features. The IBS determiner 18 then applies Equation 21 to the values of the 26 ultimate features. Equation 21 is copied below for convenience:

$$f = \frac{1}{1 + {e}^{- Σ {c}_{{i}^{x} i}}} \qquad ( Eq . 21 )$$

In doing so, the IBS determiner 18 associates each feature obtained from the feature extractor 16 with the weighting coefficient associated with that feature (see Table 1) using Equation 20 (copied below for convenience), where ‘xᵢ’ represents one of the features, ‘i’ is an integer from 1 to 26, and ‘cᵢ’ is a weighting coefficient associated with each one of the features ‘xᵢ’:

$${\sum }_{n}^{i} {c}_{i } {x}_{i} = {c}_{1} {x}_{1} + {c}_{2} {x}_{2} … + {c}_{n} {x}_{n} \qquad ( Eq . 20 )$$

The IBS determiner 18 also comprises threshold storage 40 for storing a threshold against which the IBS determiner compares the calculated value of ‘f’. In this example, the threshold storage 40 stores a threshold of 0.5, such that if the IBS determiner 18 determines that f>0.5 the subject is likely to have IBS, and conversely if the IBS determiner 18 determines that f<0.5 the subject is not likely to have IBS. It will be appreciated that the higher the value of ‘f’ the more likely the subject has IBS, and the lower the value of ‘f’ the less likely the subject has IBS. The IBS determiner 18 thus generates an index value that indicates the likelihood of IBS.

Similarly, in the example when determining a likelihood of IBD versus healthy bowels, the reference parameters are the weighting coefficients listed in Table 2 above, which were derived from the process of selecting the optimum hybrid features. The IBD determiner 18 applies Equation 21 to the values of the 44 ultimate features and in doing so, the IBD determiner 18 associates each feature obtained from the feature extractor 16 with the weighting coefficient associated with that feature (see Table 2) using Equation 20, where ‘i’ is an integer from 1 to 44. The IBD determiner 18 also comprises threshold storage 40 for storing a threshold against which the IBD determiner compares the calculated value of ‘f’. In this example, similarly to the IBS example, the threshold storage 40 stores a threshold of 0.5, such that if the IBD determiner 18 determines that f>0.5 the subject is likely to have IBD, and conversely if the IBD determiner 18 determines that f<0.5 the subject is not likely to have IBD. It will be appreciated that the higher the value of ‘f’ the more likely the subject has IBD, and the lower the value of ‘f’ the less likely the subject has IBD. The IBD determiner 18 thus generates an index value that indicates the likelihood of IBD.

In a third example, the GI condition determiner 18 can also be used to determine the likelihood that the subject from which the bowel sounds are obtained has IBD rather than IBS. The IBS/IBD determiner 18 then outputs an index value indicative of that likelihood of IBs versus IBD. The reference parameters are the weighting coefficients listed in Table 3 above, which were derived from the process of selecting the optimum hybrid features. The IBS/IBD determiner applies Equation 21 to the values of the 26 ultimate features and in doing so, the IBS/IBD determiner 18 associates each feature obtained from the feature extractor 16 with the weighting coefficient associated with that feature (see Table 3) using Equation 20, where ‘i’ is an integer from 1 to 26. The IBS/IBD determiner 18 also comprises threshold storage 40 for storing a threshold against which the IBS/IBD determiner compares the calculated value of ‘f’. In this example, similarly to the IBS versus healthy and IBD versus healthy examples, the threshold storage 40 stores a threshold of 0.5, and if the IBS/IBD determiner 18 determines that f>0.5 the subject is more likely to have IBD and less likely to have IBS, and conversely if the IBS/IBD determiner 18 determines that f<0.5 the subject is more likely to have IBS and less likely to have IBD. It will be appreciated that the higher the value of ‘f’ the more likely the subject has IBD, and the lower the value of ‘f’ the less likely the subject has IBD and the more likely the subject has IBS. The IBD determiner 18 thus generates an index value that indicates the likelihood of IBS versus IBD.

### Model Aggregator

A physician may choose to reach a diagnostic decision for a GI condition such as IBS for example based on the prediction derived from a single determiner, i.e. the IBS determiner, and rule out other organic diseases by concurrently carrying out stool, blood or biopsy tests.

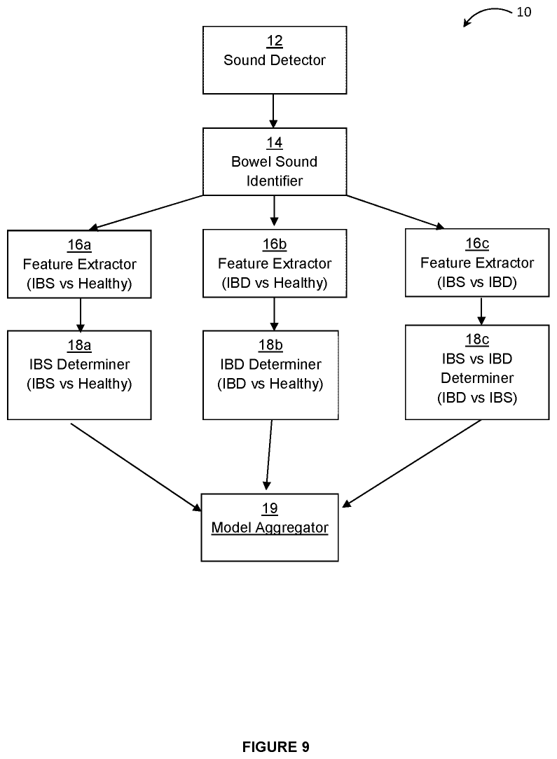

Alternatively, it would be advantageous if a physician could in practice use a single test to indicate a likelihood of a patient having either IBS, or IBD or having healthy bowels, and to differentiate between IBS and IBD. In a further embodiment, with reference to FIG. 9, the system 10 thus comprises a GI condition determiner 18 that comprises all three IBS determiner 18a, IBD determiner 18b, and IBS/IBD determiner 18c. In this embodiment, the feature extractor 16 comprises a feature extractor 16a to extract features that form part of the optimum model for determining a likelihood of IBS versus healthy bowels, a feature extractor 16b to extract features that form part of the optimum model for determining a likelihood of IBD versus healthy bowels, and a feature extractor 16c to extract features that form part of the optimum model for determining a likelihood of IBS versus IBD. The system 10 then further comprises a model aggregator 19 that facilitates the aggregation of the respective output determinations from the respective determiners 18a, 18b and 18c, and outputs an index value indicative of the following predictions:
if the IBS determiner 18a outputs an index value indicative of a determination that the patient is not likely to have IBS, and the IBD determiner 18 outputs an index value indicative of a determination that the patient is not likely to have IBD, then the model aggregator 19 outputs an index value indicative that the patient is likely to have a healthy condition, i.e. healthy bowels;
if the IBS determiner 18a outputs an index value indicative of a determination that the patient is likely to have IBS, or the IBD determiner 18b outputs an index value indicative of a determination that the subject is likely to have IBD, then the IBS/IBD determiner 18c determines a likelihood of the patient having IBS versus having IBD, and;
if the IBS/IBD determiner 18c outputs an index value indicative of a determination that the patient is likely to have IBS, then the model aggregator 19 outputs an index value indicative of a prediction of IBS; and
if the IBS/IBD determiner 18c outputs an index value indicative of a determination that the patient is likely to have IBD, then the model aggregator 19 outputs an index value indicative of a prediction of IBD.

Such a system 10 with model aggregator 19 would provide the means to differentiate between three groups, i.e. patients with IBS, patients with IBD and healthy individuals using one single test. It would constitute a non-invasive single test wherein a combination of analyses of a recording of bowel sounds allows differentiating between GI conditions with similar symptoms, such as between IBS and IBD, and healthy bowels, and would present additional clinical value.

Alternatively, the physician may choose to avoid use of a colonoscopy in the first instance and make use of the IBS versus healthy test in combination with a range of simple laboratory tests using stool and blood samples that screen for IBD (faecal calprotectin test), coeliac disease (serology) and colon cancer (feacal occult blood test) prior to making a diagnosis.

Also, if a patient has a family history or “red-flags” for inflammatory bowel disease, a physician may choose to proceed with the non-invasive test for ‘IBD versus healthy bowels’ only, which test would be an extremely useful and cost-effective screening tool, prior to confirmation of a diagnosis of IBD or other organic diseases with other tests or biopsy.

Further, if IBD has not been diagnosed following a biopsy or colonoscopy or a screening test such as the faecal calprotectin test, the physician may choose to proceed with the non-invasive test for ‘IBS versus healthy bowels’ only, or for ‘IBS versus IBD’, which would allow providing the patient with additional clinical information to confirm an IBS diagnosis and/or confirming the results of the colonoscopy/biopsy such that IBD can be ruled out as a diagnosis.

It is contemplated that the system 10 may be implemented on a single device including a belt, a plurality of sensors such as sensors V1 to V4 attached to the belt, and processing device in communication with the sensors, comprising the bowel sound identifier 14, feature extractor 16, and GI condition determiner 18. The processing device may comprise a microcontroller to control and coordinate functions of the system 10. The processing device may additionally comprise the model aggregator 19.

Alternatively, a portion of the system 10 comprising the bowel sound identifier 14, feature extractor 16 and GI condition determiner 18 may be remote from the sensors. For example, that portion of the system 10 may comprise a software program supplying instructions executable on a computing device to operate the system 10. The computing device may for example be a smartphone or other portable electronic device, or a PC. The software program may be provided in the form of a computer-readable medium.

### Method

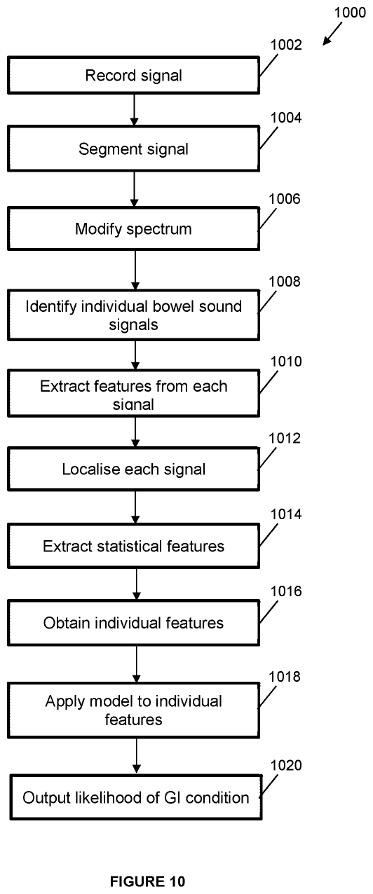

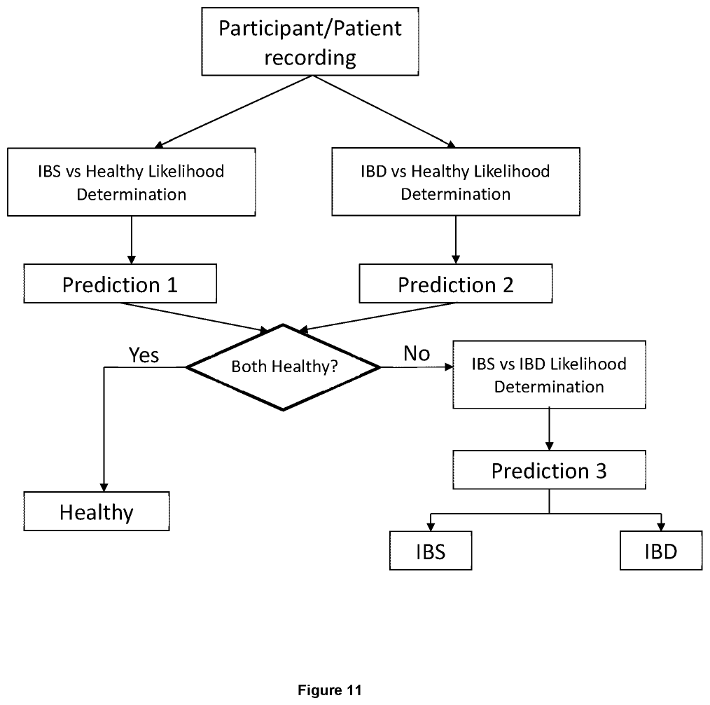

Referring to FIG. 10, a method 1000 for indicating a likelihood of a GI condition according to an embodiment of the invention is shown. The method 1000 may be carried out by the system 10 herein described. The GI condition includes IBS and IBD. However, it will be understood that the determination of a likelihood of functional GI disorder conditions other than IBS and the determination of a likelihood of GI organic diseases other than IBD are also within the scope of the present invention.

The method 1000 comprises obtaining and recording 1002 a signal representative of a plurality of bowel sounds originating from an abdominal region. As described above, the signal may be obtained by recording bowel sounds using a plurality of acoustic sensors, such as sensors V1 to V4. Each vibration sensor V1 to V4 may incorporate double transducers to allow for active noise cancellation if used in a noisy environment. Then, the recorded signal is segmented 1004 into a plurality of segments. Again, as described above, each of the segments may be 20-40 ms in length.

The segments are then modified 1006 by performing a Fourier transformation on the signal segments to obtain a frequency spectrum of the signal. Preferably, the resulting spectrums of corresponding signal segments are also modified to remove background noise. This may comprise detecting the frequency response of the sensor(s) based on the background noise and removing it from the signal spectrum.

A plurality of individual bowel sound signals is then identified 1008 by considering band energy ratios of the spectrum of each signal segment. As described above, this may comprise evaluating the BER that a signal segment has within the frequency bands: 200 Hz to 800 Hz; 600 Hz to 1000 Hz; 800 Hz to 1200 Hz; 1000 Hz to 1600 Hz; and 1600 Hz to 2000 Hz.

Features are then extracted 1010 from the identified individual bowel sound signals, such as one or more of the features listed in Table 2 above. Since this step is performed for a plurality of individual bowel sound signals, a collection of values for each feature is obtained. Accordingly, statistical distribution properties of the collection of values for each feature can be obtained. Each bowel sound signal is also localised 1014 by assigning the signal to a particular sensor V1 to V4 that produces the highest amplitude reading corresponding to the signal, as described above in relation to the signal localiser 32.

The statistical distribution properties of the collection of values for each feature are then extracted 1014. According to a specific embodiment, the statistical distribution properties include skewness and kurtosis. Further, with reference to the “Ultimate Features” column in Tables 1 to 3 above, specific distribution properties are only obtained for specific values that have been selected and prior determination has been made as to which features are most strongly associated with an indication of IBS versus healthy bowels in a first embodiment, IBD versus healthy in a second embodiment, and an indication of IBS versus IBD in a third embodiment. For example, referring to Table 1 for a determination of a likelihood of IBS versus healthy bowels, the kurtosis of the collection of amplitude values of signals assigned to V3 (feature no. 1), and the skewness of the collection of burst values of signals assigned to V3 (feature no. 4), would be extracted. Referring to Table 2 for a determination of a likelihood of IBD versus healthy bowels, the kurtosis of the collection of flatness 3000 values of signals assigned to V2 (features no. 13 and 15), and the skewness of the collection of spectral centroid values of signals assigned to V1 (features no. 11 and 12) would be extracted, for example. And referring to Table 3 for a determination of a likelihood of IBS versus IBD, the kurtosis of the collection of envelope crest factor values of signals assigned to V2 (feature no. 7) and the kurtosis of the collection of roll off values of signals assigned to V4 (feature no. 19), would be extracted, for example. Equations 23 and 24 above can be used to determine the skewness and kurtosis values. As a result, a plurality of respective values for individual or selected features is obtained at step 1016, such as values corresponding to the selected features listed in Tables 1, 2 and 3 above, respectively.

The model shown in Equation 20 can then be applied at step 1018 to the respective values for the respective selected features obtained in step 1016, which provides an output 1020 indicating the likelihood of the GI condition, i.e. IBS or IBD, versus healthy bowels. In doing so, the respective features are associated with a respective corresponding reference parameter, such as a respective weighting coefficient, stored in a library, as discussed above in relation to the GI condition determiner 18. The result is then compared to a threshold value of 0.5 to output 1020 a binary value, whereby if the result is greater than 0.5 the subject is likely to have the GI condition (IBS or IBD), and if the result is less than 0.5 the subject is not likely to have the GI condition. Alternatively, a value between ‘0’ and ‘1’ can be outputted whereby the closer the value is to ‘1’ the greater the likelihood of the GI condition. The reference parameters, as discussed in relation to the GI condition determiner 18 above, vary depending on the differentiation being made, either between IBS and healthy, IBD and healthy or between IBS and IBD.

It will be understood to persons skilled in the art of the invention that many modifications may be made without departing from the spirit and scope of the invention. For example, a different number of features may be identified by the feature identifier 30, such as only one or two features. Such features may include the ultimate features that have relatively larger weighting coefficients, such as: the kurtosis of burst ratio, the skew of the burst amount, and the skew of contraction time interval for determination of a likelihood of IBS versus healthy bowels. Moreover, it may not be necessary to take into account all of the components of the hybrid features.

Alternatively or additionally, instead of the 26 ultimate features listed in Table 1, the 44 features listed in Table 2, or the 26 features listed in Table 3, respective different combinations of features and statistical distribution properties may be used. As another example, instead of weighting coefficients, other reference parameters or properties may be used, such as a reference skew and/or kurtosis value. Furthermore, the association of features to reference parameters may comprise a direction comparison of those features to their respective reference parameters.

Further, while a single non-invasive test for IBS can be performed by a physician on a patient for whom other pathology tests, including colonoscopy and biopsy, may have been undertaken simultaneously to rule out gastrointestinal organic diseases, an embodiment of the present invention provides a method that employs a decision tree algorithm to aggregate the determinations of all three embodiments described above using the model aggregator 19 described in FIG. 9. Specifically, the model aggregator 19 allows a simultaneous determination of a likelihood of a patient having IBS versus healthy bowels and a likelihood of the patient having IBD versus healthy bowels, and if it is determined that that the patient is likely to have IBS and/or likely to have IBD, then the model aggregator 19 proceeds with a determination of a likelihood of the patient having IBS versus IBD. The decision tree algorithm then provides an overall determination as illustrated in FIG. 11, wherein if both the likelihood determination of the patient having IBS versus having healthy bowels and the likelihood determination of the patient having IBD versus having healthy bowels provide a prediction of the patient having healthy bowels, then the overall determination and output is a prediction of the patient having healthy bowels. However, if either the likelihood determination of IBS versus healthy bowels and/or the likelihood determination of IBD versus healthy bowels provide a prediction of IBS or IBD then the model aggregator proceeds to a likelihood determination of IBS versus IBD and the overall determination/output provides an indication as to whether the patient is more likely to have IBS than IBD.

It will be understood that other algorithms may alternatively be used to combine analyses of bowel sounds of patients and provide an overall determination indicative of a likelihood of a patient having either IBS, or IBD or having healthy bowels, and to differentiate between IBS and IBD. For example, other tree-based algorithms (random forest, etc.) may be used. Further, a kernel method with vector output, or a neural network method with softmax function output may also be used.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

1. A system for indicating a likelihood of a gastrointestinal (GI) condition by analysing bowel sounds, the system comprising:
a sound detector configured to detect bowel sounds and generate a corresponding signal representative of the bowel sounds;
a signal processor arranged to identify a plurality of bowel sound signals within the corresponding signal, each bowel sound signal representative of an individual bowel sound;
wherein the system is arranged to:
identify at least one feature from each of the plurality of bowel sound signals so as to produce a collection of values for the same at least one feature and obtain a distribution of the values for the same at least one feature; and
determine at least one statistical distribution property of the distribution of values for the same at least one feature, the at least one statistical distribution property being capable of at least assisting in providing an indication of the existence or non-existence of the GI condition; and
associate the at least one statistical distribution property with a reference parameter;
wherein the system is further arranged to generate an index value based on the association of the at least one statistical distribution property with the corresponding reference parameter, and compare the index value to a threshold value, in order to determine the likelihood of the GI condition.

2. The system of claim 1, wherein the at least one statistical distribution property comprises skewness and/or kurtosis.

3. (canceled)

4. The system of claim 1, wherein the system is arranged to generate the index value using the formula,
$$f = \frac{1}{1 + {e}^{- Σ {c}_{{i}^{x} i}}}$$
where ‘f’ is the index value, ‘xᵢ’ represents each one of the at least one features, ‘i’ is an integer from 1 to n, where n is the number of features, and ‘cᵢ’ represents the reference parameter associated with the feature ‘xᵢ’.

5. The system of claim 4, wherein the reference parameter is a weight value applicable to the associated statistical distribution property of the at least one identified feature.

6. The system of claim 1, wherein the GI condition comprises at least one of the following: a functional GI disorder and a GI organic disease.

7. (canceled)

8. The system of claim 6, wherein the GI condition comprises the functional GI disorder irritable bowel syndrome (IBS), and the system is arranged to determine a likelihood of IBS versus healthy bowels based on the association of the at least one statistical distribution property with a corresponding reference parameter.

9. The system of claim 6, wherein the GI condition comprises the GI organic disease inflammatory bowel disease (IBD) and the system is arranged to determine a likelihood of IBD versus healthy bowels based on the association of the at least one statistical distribution property with a corresponding reference parameter.

10. (canceled)

11. The system of claim 1, wherein the GI condition comprises a functional GI disorder and a GI organic disease and wherein the system is arranged to determine a likelihood of IBS versus IBD based on the association of the at least one statistical distribution property with a corresponding reference parameter.

12-14. (canceled)

15. The system of claim 1, wherein the at least one feature comprises, or is based on, one or more of the following: amplitude; burst amount; burst ratio; contraction interval time; higher order zero crossing; band energy ratio; spectral bandwidth double frequency; flatness; spectral centroid; energy; dynamic range; mel width; envelope crest factor; and roll off.

16. The system of claim 1, wherein the system is arranged to identify a plurality of different features from each of the plurality of bowel sounds signals and determine the likelihood of the GI condition based on a combination of the different features.

17. The system of claim 16, wherein the GI condition comprises IBS and wherein the system is arranged to determine the likelihood of IBS versus healthy bowels based on a first combination of the different features comprising at least one feature based on: burst; spectral bandwidth double frequency; contraction interval time; or higher order zero crossing.

18. The system of claim 16, wherein the GI condition comprises IBD and wherein the system is arranged to determine the likelihood of IBD versus healthy bowels based on a second combination of the different features comprising at least one feature based on: flatness 3000; or spectral centroid.

19. The system of claim 16, wherein the GI condition comprises IBS and IBD and wherein the system is arranged to determine the likelihood of IBS versus IBD based on a third combination of the different features comprising at least one feature based on: envelope crest factor; or roll off.

20. The system of claim 1, wherein the system is arranged to determine a plurality of different statistical distribution properties of the distribution of values for the at least one feature and determine the likelihood of the GI condition based on a combination of the different statistical distribution properties.

21-23. (canceled)

24. A method of indicating a likelihood of a GI condition by analysing bowel sounds, the method comprising:
obtaining a signal representative of a sound including a plurality of bowel sounds originating from an abdominal region;
identifying a plurality of bowel sound signals within the signal, each bowel sound signal representative of an individual bowel sound;
identifying at least one feature of each of the plurality of bowel sound signals within the signal so as to produce a collection of values for the same at least one feature and obtain a distribution of the values for the same at least one feature;
determining at least one statistical distribution property of the distribution of values for the same at least one feature, the at least one statistical distribution property capable of at least assisting in providing an indication of the existence or non-existence of a GI condition;
associating the at least one statistical distribution property with a reference parameter;
generating an index value based on the association of the at least one statistical distribution property with the corresponding reference parameter;
comparing the index value to a threshold value; and
determining the likelihood of the GI condition based on the comparing step.

25. The method of claim 24, wherein the GI condition comprises at least one of the following: a functional GI disorder and a GI organic disease.

26. (canceled)

27. The method of claim 25, wherein the GI condition comprises the functional GI disorder irritable bowel syndrome (IBS), and the method comprises determining a likelihood of IBS versus healthy bowels based on the association.

28-30. (canceled)

31. The method of claim 24, wherein the GI condition comprises the functional GI disorder and the GI organic disease and wherein the method further comprises simultaneously determining at least one statistical distribution property capable of at least assisting in providing an indication of the existence or non-existence of IBS, and at least one statistical distribution property capable of at least assisting in providing an indication of the existence or non-existence of IBD, whereby the method comprises simultaneously determining a likelihood of IBS versus healthy bowels and a likelihood of IBD versus healthy bowels based on respective associations of the at least one statistical distribution property with corresponding reference parameters.

32. The method of claim 31, wherein the method comprises determining a likelihood of IBS versus IBD when the index value generated indicates that IBS is more likely than healthy bowels or that IBD is more likely than healthy bowels.

33-36. (canceled)

37. A computer readable medium for storing instructions that, when executed by a computing device, causes the computer to perform the method according to claim 24.
